(19) United States
(12) Patent Application Publication
(10) Pub. No.: US 2010/0023119 A1
(43) Pub. Date: Jan. 28, 2010

(54) Valve Mold and Prosthesis for Mammalian Systems

(75) Inventors: Joon Hock Yeo, Singapore (SG); Kee Hiang Lim, Singapore (SG); Wolfgang Anton Goetz, Regensburg (DE)

Correspondence Address: KNOBBE MARTENS OLSON & BEAR LLP, 2040 MAIN STREET, FOURTEENTH FLOOR, IRVINE, CA, 92614, US

(73) Assignee: NANYANG TECHNOLOGICAL UNIVERSITY, Singapore (SG)

(21) Appl. No.: 12/065,892

(22) PCT Filed: Sep. 6, 2006
(86) PCT No.: PCT/IB2006/003495

§ 371 Date: Sep. 1, 2009

Related U.S. Application Data
(60) Provisional application No. 60/714,629, filed on Sep. 6, 2005.

(51) Int. Cl. A61F 2/24; B28B 11/14

(52) U.S. Cl. 623/2.14; 425/142; 425/500; 425/394

(57) ABSTRACT

A valve mold for a prosthetic valved conduit includes two thin film transparent plastic templates having the molding shape of the valve to sandwich biological tissue membrane to form the valve. In another embodiment, a valve mold template has a 3-dimensional geometry which, when folded and sutured to form a close-loop, resembles the geometry of a native aortic valve. The attachment side of a template designed in accordance with the principles of the invention may have a slightly undulating edge, according to one embodiment.

### FIELD OF THE INVENTION

The present invention relates to valve molds and prostheses for mammalian systems in general and more particularly, to a valve mold based on an improved template design and improved prosthesis formed therewith.

### BACKGROUND OF THE INVENTION

The listing or discussion of a prior-published document in this specification should not necessarily be taken as an acknowledgement that the document is part of the state of the art or is common general knowledge. All documents listed are hereby incorporated herein by reference.

Valves in mammalian systems are one-way valves that maintain the forward movement of the blood. The largest valves in mammalian systems include the aortic valve and the pulmonary valve. The treatment of valve disease is to either surgically repair or replace the damaged valve with a prosthesis. The two main types of valve substitutes are mechanical prosthesis and tissue valves. The main advantages of the mechanical prosthesis are their structural durability, availability, easy surgical implantation, guaranteed competence and excellent hemodynamic performance. The main drawbacks of such mechanical valves is the need for maintaining the patient permanently and adequately anticoagulated, which entails the risk of thromboembolism or hemorrhage. These problems are most significant in children, women of childbearing age and in patients living in areas of the world where drug availability or patient education is suboptimal. Finally, such mechanical valves tend to be expensive, and therefore beyond the economic possibilities for many patients.

Over the years, advances in tissue valves have been made, including valves made from porcine, bovine pericardium and homograft. These valves can either be stented or stentless. Initially tissue valve replacements were stented either porcine valves supported by a metallic or plastic stent or bovine pericardium valve supported by a metallic or plastic stent. One significant advantage of these stented tissue valves over mechanical valves is that they do not need permanent anticoagulation. The main disadvantage of stented tissue valves is limited durability. High stresses have been found along the edge of the rigid stent mounting area in these valves. The most widely used tissue valve is the porcine/bovine aortic valve treated with glutaraldehyde and supported within a metallic or plastic stent with a cloth flange for suturing it to the patient. These valves, or bioprosthesis, do not require anticoagulation, but have a limited durability in younger patients. As such, their use is typically limited to patients above 65 years of age.

The mold design disclosed in U.S. Pat. No. 6,491,511 (“Duran”) is used to shape biological tissue membrane, to form a reconstituted heart valve for replacement that closely resembles the native valve. Autologous tissue valves are made from the patient's own tissue and can be homologous or heterologous. One significant advantage of autologous tissue valves over other tissue valves is the lack of immune response from the body. However, the valves produced in accordance with Duran, like all stentless bioprosthesis, are difficult to work and can require on the order of 50-60 minutes to implant. The Duran mold has a pronounced curvature along the side to be sutured. In fact, the curvature of the mating surface of the cusps is described in Duran as an ellipse defined by the equation x²/a²+y²/b²=1, where ‘a’ has a value greater than zero and less than 22.0 (0<a<22.0), and ‘b’ has a value greater than zero and less than 14.0 (0<b<14.0). This highly curved attachment line requires the use of three continuous running sutures—one for each of the three cusp or valve leaflets. This method of suturing is not only clinically demanding, but also time consuming. Thus, there is a need in the art for an improved aortic valve mold and a prosthesis formed therewith which overcomes one or more of the aforementioned drawbacks.

### SUMMARY OF THE INVENTION

The present invention relates to a valve mold and prosthesis for mammalian systems using an improved design. In one embodiment, a mold for forming a replacement tissue valve includes a first template comprised of a thin film polymer having three continuously linked cusps formed thereon in which the first template includes a first lower undulating side that is longer than a first upper side. The mold further includes a second template comprised of a thin film polymer having three continuously linked cusps formed thereon in which the second template includes a second lower undulating side that is longer than a second upper side. In one embodiment, the first and second templates interlock to accommodate a membrane interposed there between that is to be formed into the replacement tissue value.

Other embodiments are disclosed and claimed herein.

### BRIEF DESCRIPTION OF THE DRAWINGS

FIG. 1 is a perspective view of one embodiment of a valve mold template designed in accordance with the principles of the invention;

FIG. 2 is another perspective view of the valve mold template of FIG. 1;

FIGS. 3A-3D depict one embodiment of a single-piece mold template incorporating both positive and negative templates, designed in accordance with the principles of the invention;

FIG. 4A is a side view of one embodiment of a valve mold template designed in accordance with the principles of the invention;

FIG. 4B is a cross-sectional view of one embodiment of top and bottom templates interconnected in accordance with the principles of the invention;

FIG. 5 is a photograph of a perspective view of a sizer used to determine the size of a required valve mold, according to one embodiment;

FIGS. 6A-6C depict a vascular prosthesis based on an embodiment of the invention;

FIG. 7A-7B depict the orientation between pericardium and one embodiment of a valve mold of the invention; and

FIG. 8 depicts one embodiment of a process for forming the vascular prosthesis of FIGS. 6A-6C using the valve mold template of FIG. 1.

### DETAILED DESCRIPTION OF THE EXEMPLARY EMBODIMENTS

One aspect of the invention relates to a mold for replacement valves and instrumentation for the construction in the operating room of a sigmoid (for aortic or pulmonary) valve using a biological tissue membrane, such as the patient's own pericardium either autologous or porcine, bovine or other mammalian pericardium. In one embodiment, autologous pericardium decreases the incidence of immunological rejection of the valve.

Another aspect of the invention is to provide a valve mold design where a conical tube is anchored at a wider end to the inflow orifice, and its smaller or outflow end is minimally anchored to the walls of the great vessels. In one embodiment, the molds of the present invention comprises two thin film plastic templates having the molding shape of the valve to sandwich the pericardium to form the valve. After the pericardium is placed between the two thin film plastic templates, the template-pericardium sandwich can be trimmed or cut to the appropriate size and/or shape. In one embodiment, the lateral aspects of the trimmed pericardium can be joined together to form a truncated cone with a base (inflow) orifice that is larger than its upper (outflow) orifice. The base orifice may correspond to the base of the new valve, while the outflow orifice has three slight curvatures corresponding to the three free edges of the new prosthesis. The three points joining the three slight curvatures may correspond to the three commissures of the new prosthesis.

The general implantation time of a reconstructed aortic valve using Duran's 3-dimensional template would take about 60 minutes. Recent animal trials show that, when performed in accordance with the principles of the invention, the implantation process is simplified by allowing the inflow orifice of the reconstructed valve (using biological membrane) to be suture around the native aortic annulus and the commissures (outflow orifice) sutured at 3 points of the aortic wall using, for example, a U-stitch. Using this approach, the implantation time may be significantly reduced to 30 minutes or less. Reduction in implantation time directly reduces the cardio-pulmonary bypass time, which is a critical variable determining the outcome of every cardiac valve surgery.

In another embodiment, or in addition to one or more of the previous embodiments, a valve mold template may be a 3-dimensional geometry which, when folded and sutured to form a close-loop, resembles the geometry of a native valve. The attachment line of a template designed in accordance with the principles of the invention may have a slightly undulating edge.

Another aspect of the invention is to provide a valve mold design that includes three cusps which, when folded, create three “bulges” that resemble the native valve's three cusps. In one embodiment, these three cusps are linked continuously in that there is no narrow band or other flat connecting strip separating them. Heretofore, heart valve mold designs have interspersed connecting strips between the cusps to provide flat areas in order to suture the prosthetic heart valve to the aortic root of the patient. However, the heart valve mold design may similarly include interspersed connecting strips between three adjacent cusps.

In another embodiment, there may be a narrow band only at the outer end of the valve mold which is usable for suturing. Thus, when folded, the three cusps may be formed into a reconstructed valve with no connecting strips for suturing between the cusps.

Another aspect of the invention is to provide a valve mode design which, when extended, exhibits a fan-like design in that the length of the valve mold along the base (inflow) orifice is more than the length of the valve mold along the upper (outflow) orifice. In another embodiment, a valve mold design of the invention may be designed for a three-point attachment of the valve's upper orifice.

In another embodiment, a template designed in accordance with the principles of the invention may be made of a sufficiently thin polymer such that it is transparent allowing a clear view of the biological membrane. This thin polymer design may also allow an operator to effectively trim or cut through the pericardium-template sandwich to a desired size and/or shape. Thus, a single template design may be used once. Moreover, the thin film polymer valve mold may also include markings and guidelines that effectively guide the surgeon to trim/cut/fashion the membrane template-sandwich between the valve mold templates to the desired geometry. The thin and transparent mold allows the surgeon to easily cut through the sandwich with scissors or a scalpel directly.

Yet another aspect of the invention is to provide a single piece template that incorporates both the male/female (positive/negative) templates of the 3-dimensional geometry of the intended reconstructed valve.

Still another aspect of the invention is a vascular prosthesis constructed using a mold template as described herein.

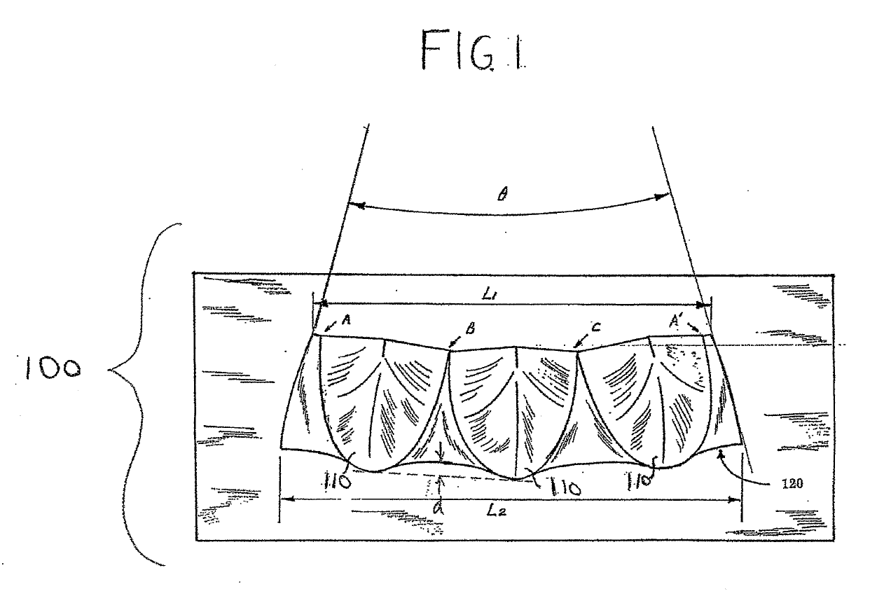

Referring now to the figures, FIG. 1 depicts a valve template 100 in a 3-dimensional geometry which, when folded and sutured to form a close-loop, resembles the geometry of a native aortic valve. When folded, the three continuously linked cusps 110 create three “bulges” that resemble the native valve's three cusps. It should be noted that, in this embodiment, the continuously linked cusps 110 do not have the typical connecting strips between the cusps which have heretofore been used to provide flat areas for suturing the heart valve to the aortic root of the patient.

Moreover, the attachment side of template 100 (which is side 120) is a slightly undulating edge, according to one embodiment. In another embodiment, the undulations of side 120, as measured by ‘d’, range from 0.0001 mm to 20 mm and preferably in the range of 1 to 15 mm. The slightly undulating side 120 may enable the production of a re-constructed valve that is nearly circular. This circular feature of the attachment line (corresponding to side 120) of the re-constructed valve may be advantageous since it enables a more effective suturing technique to be used. In particular, the technique of multiple interrupted suturing can be used to attach a replacement valve constructed with template 100. Molds of the prior art have highly non-circular attachment lines, necessitating the use of continuous running sutures, which is clinically demanding and time consuming.

In one embodiment, an aortic valve consistent with the principles of the invention includes a tri-leaflet, reed-like structure. In this fashion, the truncated cone may be supported at both ends by the two cables that maintain a circular flow and outflow orifices of the prosthesis. In one embodiment, these threads are removable after implantation. In such a case, after removal of the patient's diseased valve cusps, the base of the cylinder may be sutured to the patient's aortic base. The three commissural points of the cone in its outflow orifice may then be sutured with a pledget to the patient's aortic wall beyond the patient's own commissures. In one embodiment, a completely stentless prosthesis may be achieved by cutting and pulling out the inflow and outflow cords.

It should further be appreciated that the template 100 of FIG. 1 may be used in conjunction with a second template (not shown) to sandwich and form a patient's pericardium to be used as a replacement aortic valve. In the embodiment of FIG. 1, a length (L2) of the template 100 along the attachment line is longer than a length (L1) of the template 100 along the opposite side. This produces a fan-like appearance to the template 100 when in the extended position. This also means the sides of the template 100 can be defined by a fan angle θ, as shown in FIG. 1. In one embodiment, the fan angle θ may vary from about 10 degrees to about 60 degrees. In another embodiment, the difference between L1 and L2 may range from about 1 mm to about 30 mm.

The lengths L1 and L2 of template 100 will depend on the size of the valve to be implanted. In one embodiment, when used as an aortic mold template, the final diameter of the valve will range from approximately 9 mm to approximately 35 mm. However, it should equally be appreciated that the diameter may be smaller or larger, depending on the size of the valve needed. It can also be customized for larger or smaller hearts such as in the case of an infant or for larger hearts if required.

The template 100 of FIG. 1 is designed for a three-point attachment of the valve's upper orifice. That is, points A, B, C and A′ are usable to attach the replacement valve at the top of the commissural posts. It should be noted that when the template 100 is folded, points A and A′ meet, thereby becoming the same point of attachment. Points B and C represent the other two points of the three-point attachment design.

It should be appreciated that template 100 may be made of a sufficiently thin polymer such that it is transparent allowing a clear view of the biological membrane (including autologous pericardium) when it is “sandwiched” between a pair of the valve mold templates. A clear view of the biological membrane when sandwiched between the valve molds may be desirable because the surgeon would be able to ensure that the membrane is evenly spread, with virtually no air being trapped under or above the membrane to ensure complete treatment of the membrane, and that there is no overlapping or crumpling of the membrane within the valve mold. Any movement of the membrane (even during the trimming process) can be readily observed to allow the surgeon to make any necessary adjustments. Template 100 can be manufactured from a number of plastic materials, including high density polyethylene, polypropylene, polyesters, polyamides and other suitable plastic materials. In one embodiment, template 100 may be manufactured using an injection molding process or vacuum thermal forming or any other plastic-forming process.

In another embodiment, the thin film polymer valve mold template may be made of sufficiently thin film to allow easy trimming, and yet rigid enough to hold the membrane and to be molded according to the desired geometry provided by the valve mold. In contrast, the mold disclosed in Duran required the operator to trim the membrane along the peripheral of the valve mold template, which made it difficult to trim at narrow angles or in small radius regions.

In yet another embodiment, the thin film polymer valve mold may include markings and guidelines that effectively guide the surgeon to trim/cut/fashion the membrane sandwiched between the valve mold templates to the desired geometry.

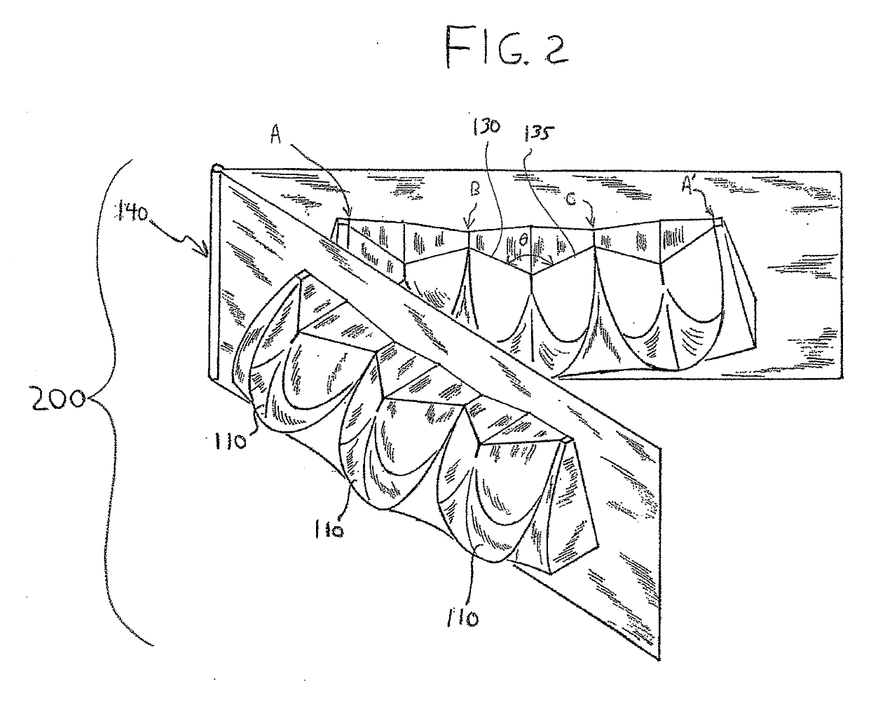

Referring now to FIG. 2, illustrated is one embodiment of a single-piece template 200 that incorporates both the male/female (positive/negative) templates of the 3-dimensional geometry of the intended reconstructed valve. As shown, the positive template and negative template are connected along hinge 140.

Each cusp 110 is designed with a cusp angle θ formed by lines 130 and 135. In one embodiment, cusp angle θ may range from approximately 100 degrees to approximately 160 degree. Proper selection of the cusp angle θ will ensure that the three leaflets of the heart valve are molded and assembled in accordance with the invention, these leaflets should contact each other to properly close the heart valve during diastolic phase. In this embodiment, the three cusps 110 are continuously linked in that there is essentially no spacing or distance between the cusps 110, as shown in FIG. 2.

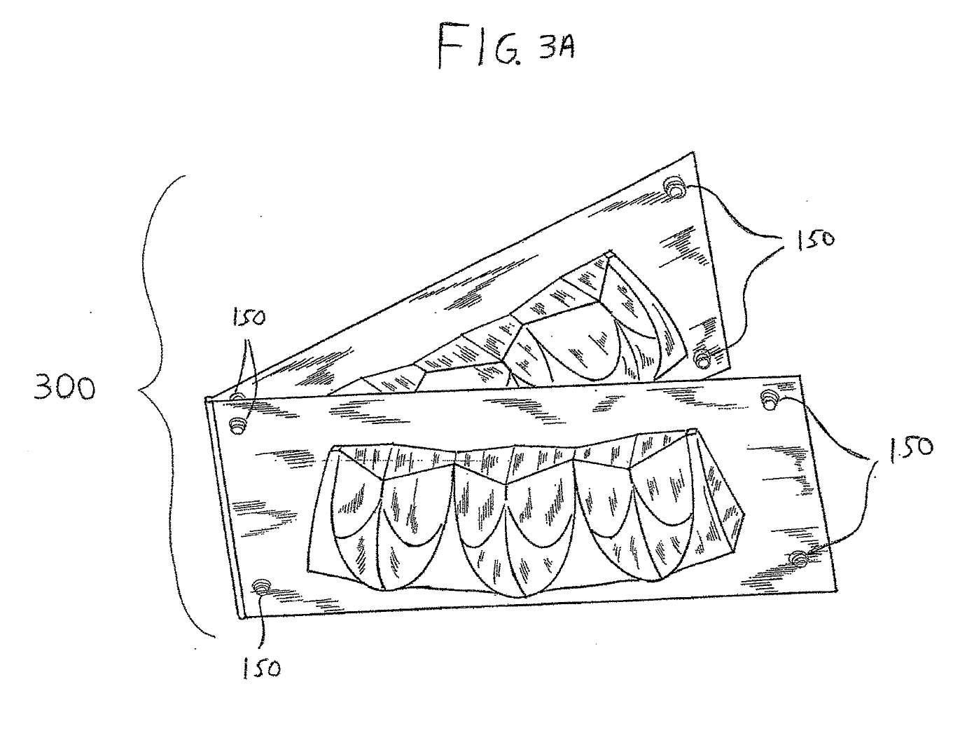

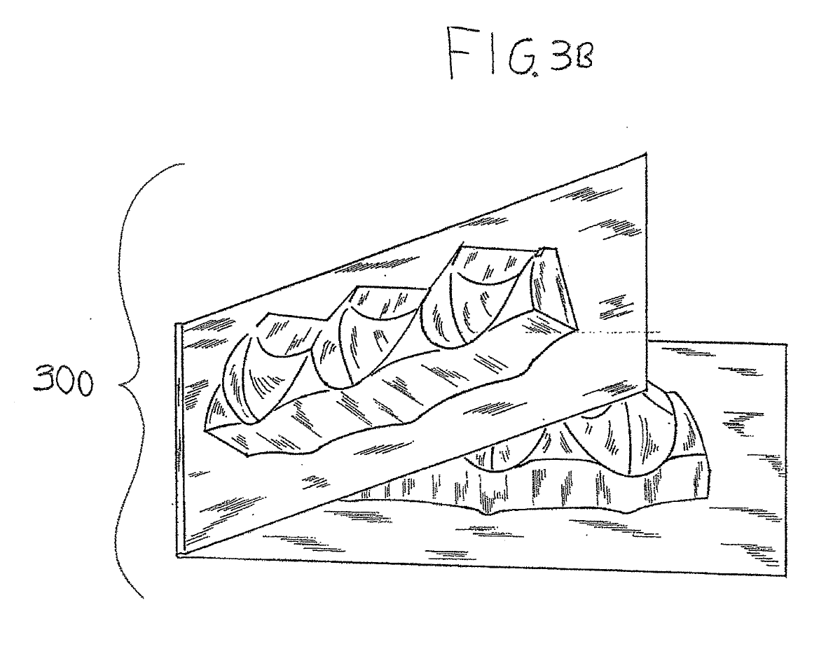

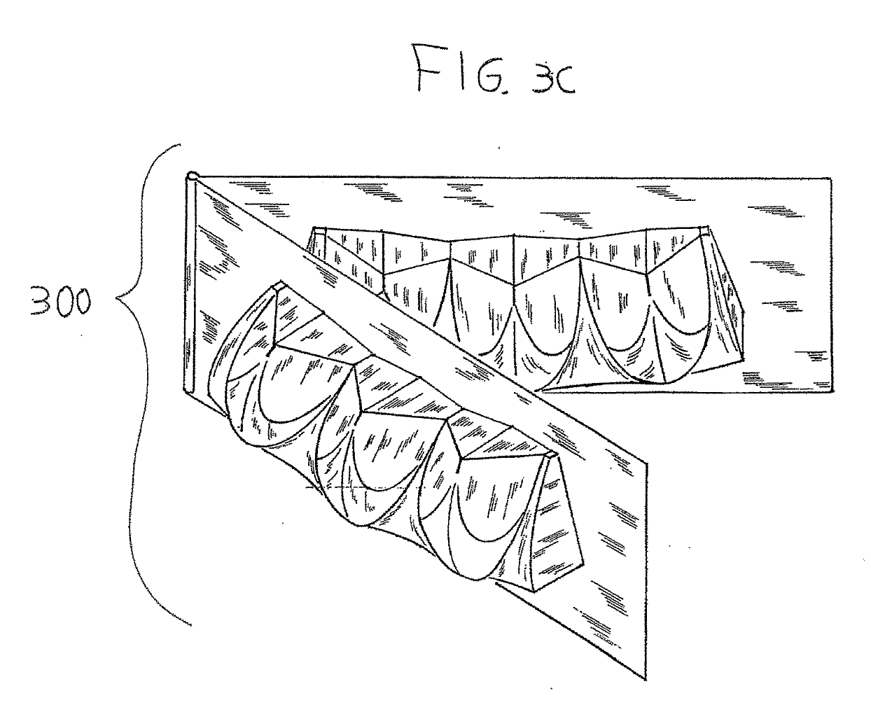

FIGS. 3A-3D depict one embodiment of a single piece template 300 that incorporates both the male/female (positive/negative) templates of the 3-dimensional geometry of the intended reconstructed valve. FIGS. 3A-3C depict the single-piece template 300 in a substantially open position, while FIG. 3D depicts the single-piece template 300 in a substantially closed position. Rather than requiring two individual and separated valve molds or templates, the embodiment of FIGS. 3A-3D simplify the molding process by removing the possibility that a positive template may be put over a negative template, rather than a negative template being put over a positive template.

The single piece template 300 may optionally include a series of inter-locking and matching stubs 150 on both the male/female (positive/negative) sides, as shown in FIGS. 3A and 3D. In one embodiment, these stubs 150 may allow the membrane to be firmly secured between the templates, such as during the treatment process. It should further be appreciated that the male/female (positive/negative) sides of single-piece template 300 may be hinged from any of the four lateral sides. The templates 300 of FIGS. 3B and 3C, which are depicted without the optional stubs 150, may be secured using one or more clamps, clips or numerous other known securing means as would be evident to one skilled in the art.

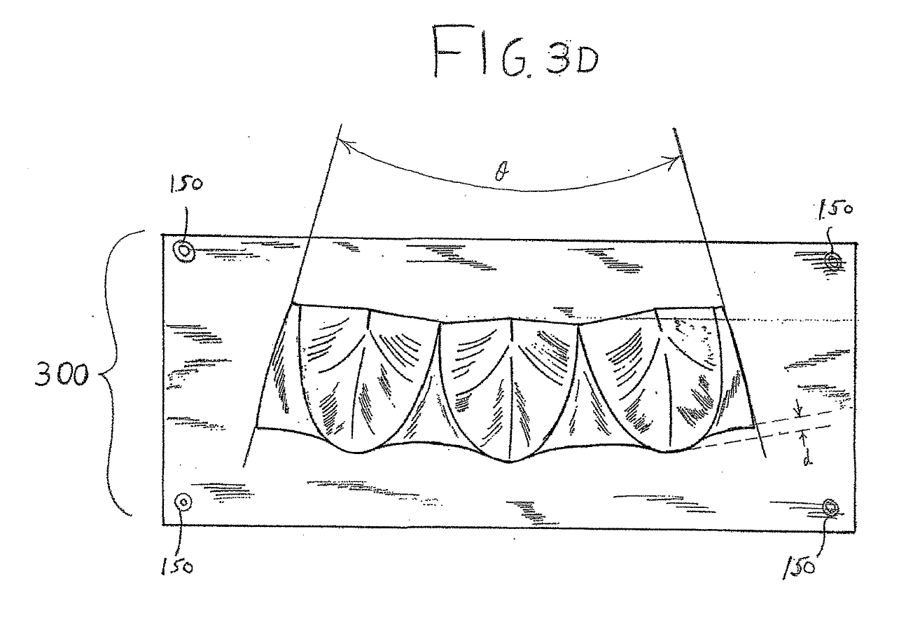

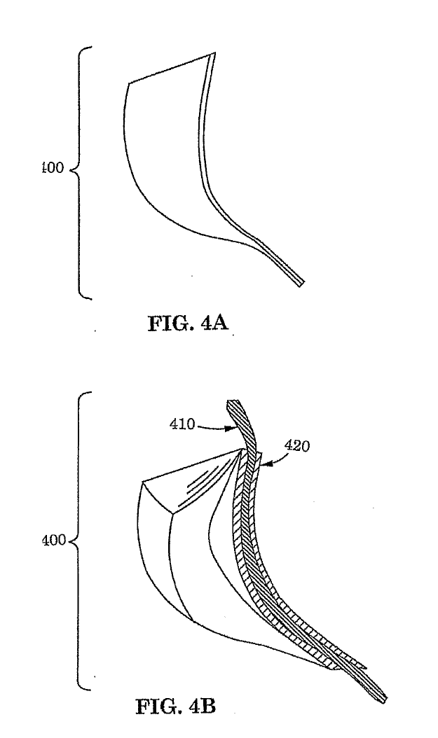

FIG. 4A depicts a side view of a single template 400, designed in accordance with the principles of the invention. In one embodiment, template 400 is a female template. FIG. 4B, on the other hand, is a cross-sectional view of template 400 being used to sandwich a membrane 410 with a male template 420. In one embodiment, membrane 410 is fresh pericardium obtained from the patient in question. This membrane may then be stretched into place and sandwiched between a female and male template (e.g., template 400 and 420). Thereafter, the mold templates 400 and 420, along with membrane 410, may undergo the treatment process by being submerged in a tanning solution, such as glutaraldehyde, in order to properly treat the membrane 410. This treatment may be used to render the tissue temporarily more rigid, thereby improving its workability and can be done before and/or during the molding process. In one embodiment, the membrane 410 is treated from 4 to 10 minutes, depending on the concentration of the solution.

After the membrane has been treated, the excess membrane 410 may be trimmed along the edges of the templates 400 and 420. However, if where the templates are not of the required shape and/or size, the templates 400 and 420 themselves, along with the sandwiched membrane 410, may all be trimmed to a specific size and/or shape. This would enable a surgeon to use a universally designed template, yet still be able to tailor it to the needs of the specific patient. In this case, it should further be appreciated that the templates may be made of thin plastic having a thickness on the order of less than approximately 0.5 mm.

After the trimming process, the two loose ends of the trimmed tissue may then be attached to one another to form the prosthetic valve. Thereafter, the replacement heart valve may be sutured into the aortic or pulmonary valve root, which in one embodiment is done using a multiple interrupted suturing process. Once implanted, the autologous tissue will slowly regain the consistency of the surrounding tissue, and will function in a normal opening/closing valve fashion.

Molds of the invention were tested in two test groups of sheep. The first group was tested with prosthetic valves made with the sheep's autologous pericardium using a preferred embodiment of the valve mold of the invention. The constructed valve was placed in the pulmonary valve root. The valve implanted was constructed intra-operatively as an autologous pericardial heart valve made of the sheep's own pericardium, treated for 8 minutes with buffered Glutaraldehyde. In this first group, the valve was implanted in 6 sheep in pulmonary position and in 6 sheep in aortic position under cardiopulmonary bypass. After implant, all valves were immediately competent and no regurgitation was detectable. The hemodynamic study showed very low transvalvular pressure gradients after implantation. After six month, the sheep of the pulmonary implants were sacrificed showing promising results with competent valves. At time of sacrifice transvalvular gradient was 4.5+/−1.9 mmHg. There was no valvular or paravalvular leak, the leaflets were pliable and thin. Histology showed no tear or rupture at the Single Point Attached Commissures (SPAC). Subsequently the SPAC valve was implanted as autologous pericardial valve in the same manner in aortic position.

The second group consisted of 20 Merrino sheet in which molded autologous aortic valve prostheses were implanted. The valve prostheses were constructed from the sheep pericardium in less than 15 min. In each case, the native valve was removed and a prosthesis was implanted in less than 30 minutes using cardio pulmonary bypass. Epicardial echo demonstrated well working valve prostheses with insignificant regurgitation. Postmortem revealed all valve leaflets to be pliable with minor calcification in a few leaflets. Except for one incidence, the commissures were reliably anchored to the aortic wall. After changing the implantation technique by adding a pledged outside of the aorta at SPAC, no more disruption of SPAC occurred. Implants in this second group of sheep showed overall excellent results that appear to be as good a commercially available valve prostheses. In the A-Series, the commissures were implanted at the aortic wall with a 4/0 suture. In this series, one torn commissure was found. The suture at the commissure had been cutting through the aortic wall, the suture loop was still found to be anchored at the free-floating commissure and the pericardial leaflet structure was still intact at this location. Due to this incidence, the implantation technique of the commissures was changed by tying the 4/0 suture over a pledged outside of the aorta. Subsequently in the next series (H-series) all commissures were found to be intact, with no tears or alterations of the aortic wall and the pericardial leaflet.

According to another aspect of the invention, the mold may be prepared together as a kit that includes a sizer for accurate measurement of the diameter of a valve to the correct size required of a valve that is to be replaced and/or a tanning solution. These sizers may be any design that fit inside or across the valve root as long as they are capable of determining the diameter of the valve that needs to be replaced. One embodiment of the sizer 500 can be seen in FIG. 5. In this embodiment, a round head 510 is attached to a handle portion 520 to allow it to be quickly placed at the valve root by the operating team for determining the valve diameter of a ready made valve or the valve mold of the invention needed for preparing a valve.

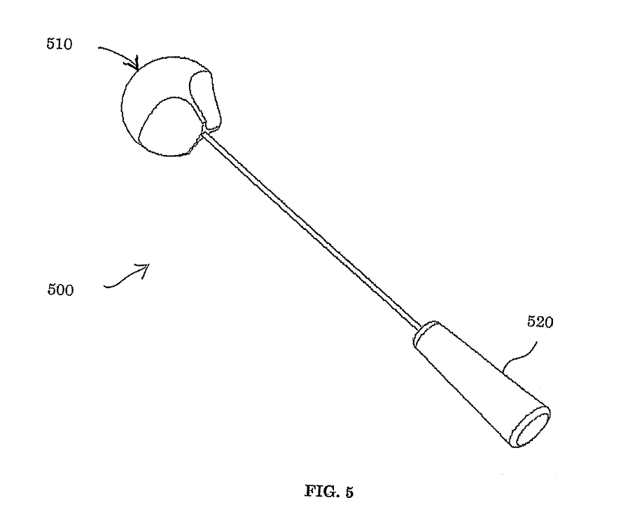

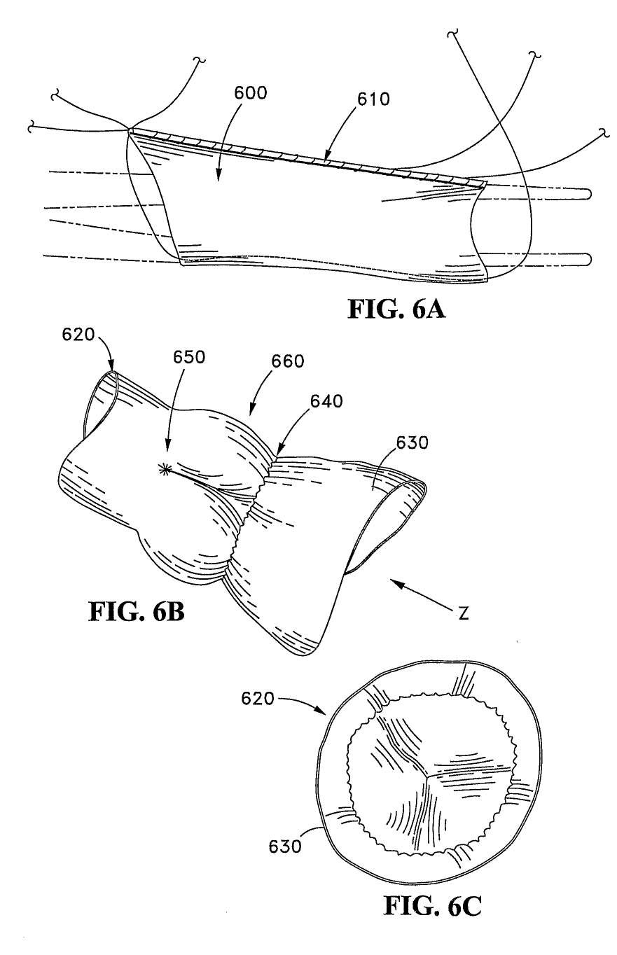

Referring now to FIGS. 6A-6C, depicted is a prosthetic pulmonary conduit formed using a valve mold template (e.g., template 100) consistent with the principles of the invention. In particular, FIG. 6A depicts a prosthetic pulmonary conduit 600 in which the 3-dimensional shaped pericardial flap has been then sutured together along the lateral side 610 in a way that it will form a vascular graft. The conduit 600 may optionally include sinus bulges that correspond to each valve cusp. FIG. 6B depicts another embodiment of a valved conduit 620 in which an additional strip of pericardium 630 has been sutured to the base of the valved conduit 620 along the line 640. The lower part of the valved conduit may then be sutured to the right ventricular outflow tract, while the upper part may be sutured to the pulmonary trunk, as previously described. FIG. 6B further depicts the placement of a suture 650, which may be one of the three sutures used in the aortic wall. The valved conduit 620 may optionally have sinus bulges 660 (Sinus of Valsalva) corresponding to each of the three valve cusps. Finally, FIG. 6C depicts the valved conduit 620 of FIG. 6B when viewed from the Z direction, with the strip of pericardium 630 forming a wider base to accommodate the right ventricular outflow tract.

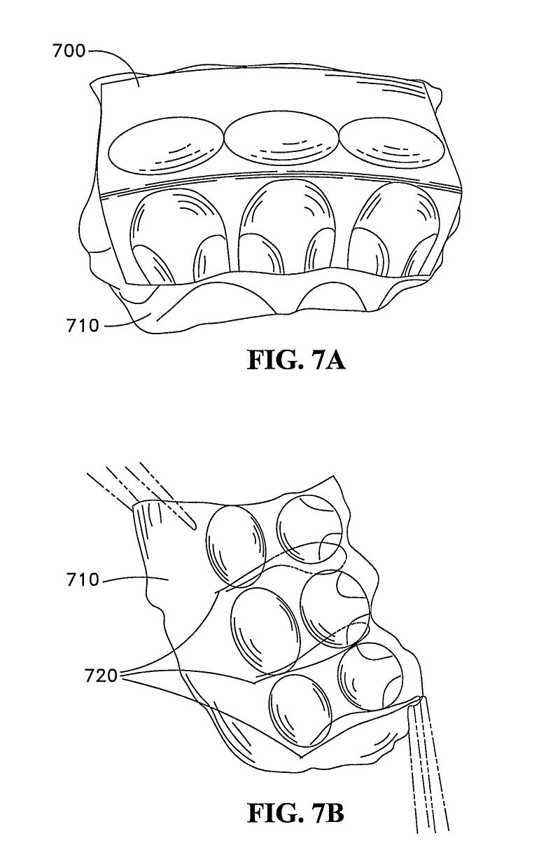

FIGS. 7A-7B depict the interaction or orientation between one embodiment of a valve mold 700 of the invention, and a piece of harvested pericardium 710. With respect to FIG. 7A, the pericardium 710 is positioned to be sandwiched between the individual templates of the mold 700. FIG. 7B shows the pericardium 710 of FIG. 7A after being trimmed and treated, and after sutures 720 have been placed at the commissures of the valve leaflets and passed through the conduit wall.

Continuing to refer to FIGS. 7A-7B, unlike the three continuously links cusps depicted in FIG. 1, the valve mold 700 includes a narrow band or other flat connecting strip separating the three adjacent cusps.

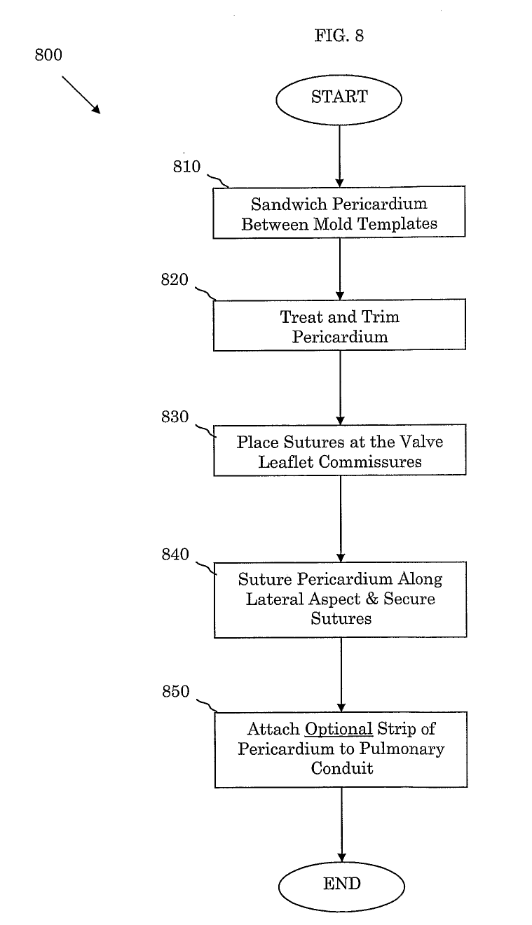

FIG. 8 illustrates one embodiment of a process 800 for forming a prosthetic pulmonary conduit (e.g., conduit 620) using a mold template designed in accordance with the principles of the invention. Process 800 begins at block 810 with a piece of harvested pericardium being sandwiched between mold templates of the invention (e.g., templates 400 and 420). Thereafter, at block 820 the pericardium is trimmed and tanned with Glutaraldehyde, for example. This step will form the pericardium into a 3-dimensional shape, which makes it possible to handle the pericardium and to manufacture a valved conduit out of s single piece of pericardium.

At block 830, sutures may be placed at the commissures of the valve leaflets and passed through the conduit wall at three equidistant points at the sino-tubular junction. The 3-dimensional shaped pericardial flap may then be sutured together along the lateral side in a way that it will form a vascular graft at block 840.

In one embodiment, the commissural sutures may be pulled, thereby inverting the leaflets into the conduit. The sutures may then be securely tied outside of the conduit wall, forming a three-leaflet valve.

Process 800 may then proceed to block 850 where an optional strip of pericardium may be sutured to the base of the valved conduit to match the right ventricular outflow tract, if necessary. The lower part of the completed valved conduit is ready to be sutured to the right ventricular outflow tract, while the upper part may be sutured to the pulmonary trunk.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

What is claimed is:

1. A mold for forming a replacement tissue valve comprising:
a first template comprised of a thin film polymer having three continuously linked cusps formed thereon, wherein said first template includes a first lower undulating side that is longer than a first upper side; and
a second template comprised of a thin film polymer having three continuously linked cusps formed thereon, wherein said second template includes a second lower undulating side that is longer than a second upper side,
wherein said first and second templates interlock to accommodate a membrane interposed there between where said membrane is to be formed into said replacement tissue value.

2. The mold of claim 1, wherein said membrane has a first lateral edge and a second lateral edge, where said first and second lateral edges are joined together to form a truncated cone with an inflow orifice that is larger than an outflow orifice.

3. The mold of claim 2, wherein said first and second templates are to be trimmed to a desired size, thereby enabling said first template and second template to be usable to form replacement tissue valves of varying sizes.

4. The mold of claim 3, wherein said first and second templates further include a plurality of markings usable to guide an operator to trim said first and second templates to said desired size1

5. The mold of claim 1, wherein said first template and second template are coupled together along a hinged side.

6. The mold of claim 5, wherein said first template and second template are formed from a single piece of said thin film polymer.

7. The mold of claim 6, wherein said first template and second template further include one or more interlocking stubs usable to secure said first template and second template in an interlocking position.

8. The mold of claim 1, wherein said first and second lower undulating sides each has an undulation with a magnitude of no more than 20 mm.

9. The mold of claim 8, wherein said first and second lower undulating sides each has an undulation with a magnitude of between approximately 1 mm and 15 mm.

10. The mold of claim 1, wherein said first upper side and second upper side have a three-point attachment design corresponding to three points of attachments for said membrane to commissural posts.

11. The mold of claim 1, wherein the difference between a length of said first lower undulating side and said first upper side is from about 1 mm to about 30 mm.

12. The mold of claim 1, wherein the replacement tissue valve is a prosthetic valved conduit having a base orifice that is larger than an upper orifice, the valved conduit being formed of a pericardium membrane that is,
formed between first and second mold templates, wherein each of the first and second mold templates have three continuously linked cusps formed thereon and lower undulating sides that are longer than corresponding upper sides, wherein the upper sides correspond to the upper orifice and the lower undulating sides correspond to the base orifice,
trimmed along a perimeter of the first and second mold templates,
sutured at the commissures of three valve leaflets using commissural sutures, and
sutured along lateral sides of the pericardium membrane so as to form a vascular cone.

13. A mold for forming a replacement tissue valve comprising:
a first template portion having three continuously linked concave cusps formed thereon, wherein said first template portion includes a first lower side that is longer than a first upper side, and wherein said first lower side has a first set of undulations; and
a second template portion having three continuously linked convex cusps formed thereon, wherein said second template portion includes a second lower side that is longer than a second upper side, and wherein said second lower side has a second set of undulations that correspond to the first set of undulations,
wherein said first and second template portions interlock to accommodate a membrane interposed there between where said membrane is to be formable into said replacement tissue value.

14. The mold of claim 13, wherein said membrane has a first lateral edge and a second lateral edge, where said first and second lateral edges are joined together to form a truncated cone with an inflow orifice that is larger than an outflow orifice.

15. The mold of claim 14, wherein said first and second template portions are usable to form replacement tissue valves of varying sizes by being trimmed to a desired size.

16. The mold of claim 15, wherein said first and second templates portions further include a plurality of markings usable to guide an operator to trim said first and second template portions to said desired size.

17. The mold of claim 13, wherein said first template portion and said second template portion are coupled together along a hinged side.

18. The mold of claim 17, wherein said first template portion and second template portion are formed from a single piece of thin film polymer.

19. The mold of claim 18, wherein said first template portion and second template portion further include one or more interlocking stubs usable to secure said first template portion and second template portion in an interlocking position.

20. The mold of claim 13, wherein said first and second set of undulations have a magnitude of between approximately 1 mm and 15 mm.

21. The mold of claim 13, wherein said first upper side and second upper side have a three-point attachment design corresponding to three points of attachments for said membrane to commissural posts.

22. The mold of claim 13, wherein the difference between a length of said first lower side and said first upper side is from about 1 mm to about 30 mm.

23. The mold of claim 13, wherein the three continuously linked concave cusps comprise three adjacent concave cusps separated by first interspersed connecting strips, and wherein the three continuously linked convex cusps comprise three adjacent convex cusps separated by second interspersed connecting strip.

24. A mold for forming a replacement tissue valve comprising:
a first template portion having three adjacent concave cusps separated by first interspersed connecting strips formed thereon, wherein said first template portion includes a first lower side that is longer than a first upper side, and wherein said first lower side has a first set of undulations; and
a second template portion having three adjacent convex cusps separated by second interspersed connecting strips formed thereon, wherein said second template portion includes a second lower side that is longer than a second upper side, and wherein said second lower side has a second set of undulations that correspond to the first set of undulations,
wherein said first and second template portions interlock to accommodate a membrane interposed there between where said membrane is to be formable into said replacement tissue value.

25. A prosthetic valved conduit having a base orifice that is larger than an upper orifice, the valved conduit being formed of a pericardium membrane that is,
formed between first and second mold templates, wherein each of the first and second mold templates have three continuously linked cusps formed thereon and lower undulating sides that are longer than corresponding upper sides, wherein the upper sides correspond to the upper orifice and the lower undulating sides correspond to the base orifice,
trimmed along a perimeter of the first and second mold templates,
sutured at the commissures of three valve leaflets using commissural sutures, and
sutured along lateral sides of the pericardium membrane so as to form a vascular cone.

26. The prosthetic valved conduit of claim 25, wherein said commissural sutures are passed through a wall of the pericardium membrane at three equidistant points.

27. The prosthetic valved conduit of claim 25, wherein the pericardium membrane is further sutured with an additional strip of pericardium to said base orifice.

28. The prosthetic valved conduit of claim 25, wherein the base orifice is configured to be sutured to a mammalian right ventricular outflow tract and the upper orifice is configured to be sutured to a mammalian pulmonary trunk.

29. The prosthetic valved conduit of claim 25, wherein said lower undulating sides have undulations of no more than 20 mm.

30. A prosthetic valved conduit having a base orifice that is larger than an upper orifice, the valved conduit being formed of a pericardium membrane that is,
formed between first and second mold templates, wherein each of the first and second mold templates have three adjacent cusps formed thereon separated by interspersed connecting strips, and lower undulating sides that are longer than corresponding upper sides, wherein the upper sides correspond to the upper orifice and the lower undulating sides correspond to the base orifice,
trimmed along a perimeter of the first and second mold templates,
sutured at the commissures of three valve leaflets using commissural sutures, and
sutured along lateral sides of the pericardium membrane so as to form a vascular cone.
